(19) United States
(12) Patent Application Publication
(10) Pub. No.: US 2003/0032585 A1
(43) Pub. Date: Feb. 13, 2003

(54) Mucosal repair by TFF2 peptides

(76) Inventors: Lars Thim, Gentofte (DK); Steen Seier Poulsen, Allerod (DK)

Correspondence Address: Reza Green, Esq., Novo Nordisk of North America, Inc., Suite 6400, 405 Lexington Avenue, New York, NY, 10174-6401, US

(21) Appl. No.: 10/172,645

(22) Filed: Jun. 13, 2002

Related U.S. Application Data
(60) Provisional application No. 60/303,246, filed on Jul. 5, 2001.

(30) Foreign Application Priority Data
Jun. 14, 2001 (DK) PA 2001 00927
Feb. 4, 2002 (DK) PA 2002 00165

(51) Int. Cl. A61K 38/17

(52) U.S. Cl. 514/8; 514/12

(57) ABSTRACT

The present invention relates to the use of trefoil factor 2 (TFF2) peptides and a pharmaceutical composition comprising TFF2 peptides for increasing the viscosity of mucin in mucus layers and the repair of damaged mucus layers in the gastrointestinal tract (mouth, oesophagus, stomach, small and large intestine, colon) the respiratory passages, the eye, the urinary system (including the bladder) and the cervis uteri.

### FIELD OF THE INVENTION

The present invention relates to the use of trefoil factor 2 (TFF2) peptides and a pharmaceutical composition comprising TFF2 peptides for increasing the viscosity of mucin in mucus layers and the repair of damaged mucus layers in the gastrointestinal tract (mouth, oesophagus, stomach, small and large intestine, colon) the respiratory passages, the eye, the urinary system (including the bladder) and the cervis uteri.

### BACKGROUND OF THE INVENTION

Mammalian trefoil factors (TFFs) constitute a group of three peptides (TFF1, TFF2 and TFF3) widely distributed in the gastrointestinal tract. These peptides are characterised by containing one (TFF1 and TFF3) or two (TFF2) trefoil domains. A trefoil domain is defined as a sequence of 38 or 39 amino acid residues in which six cysteines are disulphide-linked in a 1-5, 2-4 and 3-6 configuration. The trefoil peptides are expressed in the gastrointestinal tract in a tissue specific manner. In humans TFF1 and TFF2 are expressed in mucus producing cells in the stomach and duodenum, whereas TFF3 is primarily expressed in goblet cells in the small and large intestine. In the case of gastric ulceration or inflammatory bowel disease the expression of trefoil peptides is highly upregulated. This suggest that trefoil peptides may have a repair function for damages in the gastrointestinal tract thus acting as naturally occurring healing factors. The importance of TFFs for normal mucosal function have also been investigated by two gene knock-out studies in which the genes encoding TFF1 and TFF3, respectively, were deleted by gene-targeting techniques. The TFF3 knock-out mice had impaired mucosal healing and died from extensive colitis after oral administration of dextran sulphate a situation that could be circumvented by luminal administration of recombinant TFF3. Although several studies have documented a protection or healing effect of trefoil peptides in gastric ulceration and colitis models the detailed mechanism of action is still largely unknown.

The cloning of rat and human single-domain TFF3 (ITF) and the use of this peptide in the treatment of gastrointestinal injury is described in WO 92/14837.

International patent application WO 94/17102 and U.S. Pat. No. 5,783,416 relates to human TFF2 peptides in glycosylated form, variants thereof and a method of producing TFF2 peptides in glycosylated form.

International patent application DK01/00811 relates to glycosylated Lys99-TFF2 peptides.

### SUMMARY OF THE INVENTION

The present invention is directed to pharmaceutical compositions comprising trefoil factor 2 (TFF2) peptides, with the proviso that the TFF2 peptide is not glycosylated Lys99-TFF2, and optionally, a mucin glycoprotein preparation, and to the use of such compositions for increasing the viscosity of mucin in mucus layers and for the repair of damaged mucus layers in the gastrointestinal tract (mouth, oesophagus, stomach, small and large intestine, colon) the respiratory passages, the eye, the urinary system (including the bladder) and the cervis uteri.

### BRIEF DESCRIPTION OF THE DRAWINGS

FIG. 1. The structure of human Lys99-TFF2. Disulphide bonds between Cys6-Cys104, Cys8-Cys35, Cys19-Cys34, Cys29-Cys46, Cys58-Cys84, Cys68-Cys83, Cys78-Cys95 are schematically represented.

FIG. 2. Stress versus shear rate of mucin solution alone. 2 ml of 10% (w/v) mucin I dissolved in 0.05% (w/v) sodiumazide was added 0.4 ml of water. After 30 min at 20° C. the shear stress was measured as function of shear rate using the software programme: “constant rate — Approximation to power law.

FIG. 3. Stress versus shear rate of mucin/TFF2 gel-like material. 2 ml of 10% (w/v) mucin I dissolved in 0.05% (w/v) sodiumazide was added 0.4 ml of water containing 14.1 mg glycosylated TFF2. After 30 min at 20° C. the shear stress was measured as function of shear rate using the software programme: “constant rate”. —: Shear stress (Pa); O—O: Viscosity (mPa s).

FIG. 4. Oscillatory measurement of mucin solution (a) and mucin/TFF2 gel-like material (b). 2 ml of 10% (w/v) mucin I dissolved in 0.05% (w/v) sodiumazide was added 0.4 ml of water (a) or 0.4 ml of water containing 14.1 mg glycosylated TFF2 (b). After 30 min at 200C a sinosoidally varying stress was applied and the strain response was detected at different frequencies. The complex viscosity (η*) ⋄—⋄, the elastic modulus (G′) — and the viscous modulus (G”) O—O was calculated and plotted as a function of different frequencies. Note the differences in Y-axis unit.

FIG. 5. Viscosity versus shear rate of mucin solution added increasing amount of glycosylated TFF2. 2 ml of 10% (w/v) mucin I dissolved in 0.05% (w/v) sodiumazide was added 0.4 ml of water containing 0.88 mg, 1.76 mg, 3.53 mg 7.05 mg and 14.1 mg glycosylated TFF2, respectively. After 30 min at 20° C. the viscosity was measured as function of shear rate using the software programme: “constant rate”. —: 0.88 mg; ∇—∇: 1.76 mg; Δ—Δ 3.53 mg; ⋄—⋄ : 7.05 mg and ▪—▪: 14.1 mg.

FIG. 6. Viscosity versus shear rate of three different mucins added glycosylated TFF2. 2 ml 10% (w/v) mucin I, II and III, respectively dissolved in 0.05% sodiumazide was added 7.05 mg glycosylated TFF2 dissolved in water. After 30 min at 20° C. the viscosity was measured as function of shear rate using the software programme: “constant rate”. — Mucin I; Δ—Δ Mucin II; ⋄—⋄ Mucin III.

FIG. 7. Viscosity versus shear rate at three different conditions. Experimental conditions as in FIG. 6 except that Mucin I and the TFF2 peptide was dissolved in three different solvents: Δ—Δ 0.01 N HCl; — water; ⋄—⋄ 50 mM sodiumphosphate buffer 0.1 M NaCl, pH=7.4.

FIG. 8. The effect of luminal Asn99-TFF2 in experimental colitis in rats was scored by means of a histologic colitis score (Williams K L. et al. Gastroenterology 2001 ;120:925-37).

FIG. 9. The effect of luminal Asn99-TFF2 in experimental colitis in rats. A significant effect on the overall colitis score is demonstrated in this figure.

FIG. 10. The effect of luminal Asn99-TFF2 in experimental colitis in rats. The effect was predominantly in the midsection of the colon close to the site where the Asn99-TFF2 had been introduced into the colonic lumen.

### DESCRIPTION OF THE INVENTION

The present invention relates to the use of human TFF2 peptides for improving rheological properties of mucin solutions. TFF2 peptides have by the present inventors been found to increase the viscosity and elasticity of different mucins solutions, which are correlated to physiological and pathophysiological conditions.

The present invention discloses the mechanism by which the TFF2 peptides exerts their biological activity, which are documented by a direct effect of TFF2 peptides on the viscosity and elasticity of mucin solutions. The TFF2 peptides significantly increases the viscosity of mucin solutions. The net effect is an increase in the viscosity of several times and can be visualised by the fact that the liquid mucin solution is converted into a more viscous gel-like substance.

When expressed in yeast TFF2 peptides are secreted in a glycosylated and a non-glycosylated form. The glycosylated form generates more viscous gel-like structure as compared to the non-glycosylated.

The TFF2 peptides have by the present inventors been found to be useful for increasing the viscosity and elasticity of mucus layers, which can be used in the treatment of many different indications, where abnormalities in existing mucus layers are present. The advantage over known therapies is that treatment with TFF2 peptides represent a specific treatment at the site of injury without major side effects.

For local and luminal applications TFF2 peptides can increase the viscosity and elastic properties of mucin in mucus layers, which may be useful in many different indications:

1) For the treatment of the oral mucosa. TFF2 peptides may be given alone or together with mucus-like preparations to patients with reduced secretion of saliva caused by irradiation therapy, treatment with anticholinergics or in patients with Sjögrens syndrome.

2) For increasing the viscosity of nasal secretions in rhinorrhoea in common cold or allergic rhinitis. Protection of the mucosa of respiratory tract following accidental inhalation of irritants, gases, dusts or fumes.

3) For protection of the distal part of the oesophagus against acid secretions from the stomach in reflux oesophagi's, hiatus hernia, Barrets oesophagus.

4) For the protection of the stomach against acute stress induced gastric ulcers secondary to trauma, shock, large operations, renal or lever diseases, or gastritis caused by treatment with aspirin or other NSAIDS, steroids or by alcohol.

5) For the treatment of acute or prolonged diarrhoea by increasing the viscosity of the intestinal secretions.

6) For the protection of the small intestinal and colonic mucosa in Crohns disease, ulcerative colitis or irritable bowel syndrome.

7) In eye droplets to increase the viscosity of lacrimal fluid in patients with keratoconjunctivitis sicca/Sjögren's syndrome or “dry eyes” for other reasons.

8) Local application especially in the knee joints to increase the viscosity of the synovial fluid in osteoarthritis and following joint replacement.

TFF2 peptides may also be used for parenteral applications:

Parenteral TFF2 is taken up by cells associated with stem cells in the gastrointestinal tract. It can be used for protection of the stomach against stress-induced damage and the stomach and intestine against damage following irradiation or chemotherapy or in the treatment of acute excerbations in ulcerative colitis, irritable bowel syndrome or Crohn's disease. Injected TFF2 is excreted intact in urine and may increase the defence mechanism of the urinary bladder by binding to the layer of mucopolysaccharids that coat the urothelium and thereby interfere with the adherence of bacteria in chronic bladder infections, in patients with catheter or interstitial cystitis, or interfere with the binding of urinary growth factors in papillomas or cancer of the bladder.

In a first aspect, the present invention relates to a pharmaceutical composition for increasing the viscosity of mucus layers in mammals, the composition comprising a TFF2 peptide or a pharmaceutically acceptable salt thereof; with the proviso that the TFF2 peptide is not glycosylated Lys99-TFF2.

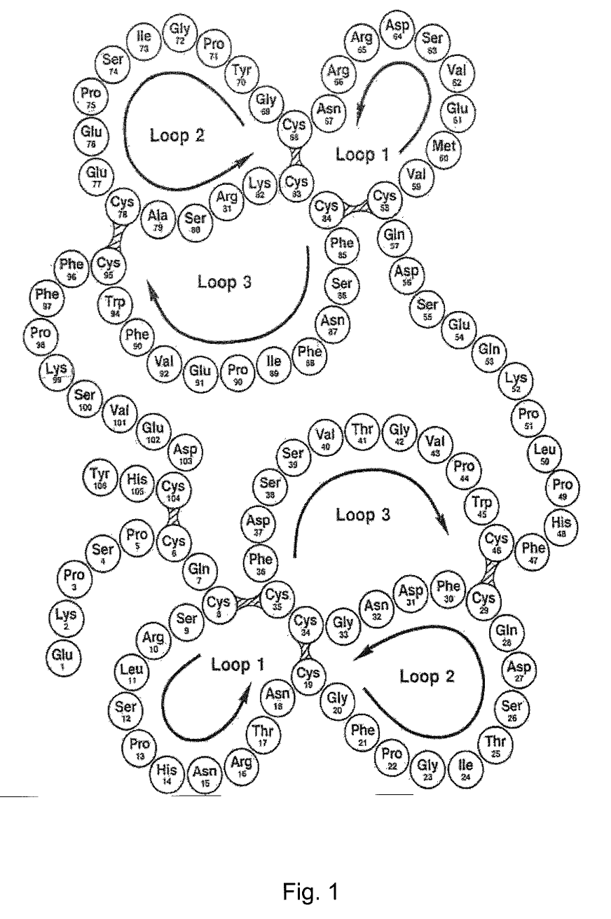

By “TFF2 peptides” or “a TFF2 peptide” is meant a protein that is substantially homologous to human TFF2, herein also denoted Lys99-TFF2 (FIG. 1). An example within this definition is the Asn99-TFF2 variant. Both Lys99-TFF2 and Asn99-TFF2 may be g lycosylated at the Asn15. The term TFF2 peptides also includes analogs of naturally occurring TFF2 peptides. Analogs can differ from naturally occurring TFF2 by amino acid sequence differences or by modifications that do not affect sequence, or by both. Analogs of the invention will generally exhibit at least 70%, more preferably 80%, more preferably 90%, and most preferably 95% or even 99%, sequence identity with a naturally occurring TFF2 sequence.

The term “glycosylated Lys99-TFF2”, as used herein, means the TFF2 peptide according to FIG. 1, which is glycosylated at the Asn15.

Modifications include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps, e.g., by exposing the polypeptide to enzymes that affect glycosylation derived from cells that normally provide such processing, e.g., mammalian glycosylation enzymes. Also embraced are versions of the same primary amino acid sequence that have phosphorylated amino acid residues, e.g., phosphotyrosine, phosphoserine, or phosphothreonine.

In addition to substantially full-length polypeptides, the term TFF2 peptide, as used herein, includes biologically active fragments of the polypeptides. As used herein, the term “fragment,” as applied to a polypeptide, will ordinarily be at least 10 contiguous amino acids, typically at least 20 contiguous amino acids, more typically at least 30 contiguous amino acids, usually at least 40 contiguous amino acids, preferably at least 50 contiguous amino acids, and most preferably at least 60 to 80 or more contiguous amino acids in length. The ability of a candidate fragment to exhibit a biological activity of a TFF2 peptide can be assessed by methods known to those skilled in the art. Also included in the term “fragment” are biologically active TFF2 peptides containing amino acids that are normally removed during protein processing, including additional amino acids that are not required for the biological activity of the polypeptide, or including additional amino acids that result from alternative mRNA splicing or alternative protein processing events.

A TFF2 peptide, including a fragment, or analog is biologically active if it exhibits a biological activity of a naturally occurring TFF2, e.g., the ability to alter viscosity or elasticity of mucin in mucus layers in a mammal.

The term “glycosylation”, as used herein, means the post-translational modification of a peptide, wherein a carbohydrate molecule is covalently attached to the peptide. The glycosylation may take place in a eucaryotic host cell, such as a yeast cell or it may be done by chemical linkage in vitro after production of the peptide in a cell, e.g. the peptide could be produced in a bacteria and glycosylated in vitro afterwards.

In a second aspect, the present invention relates to the use of a TFF2 peptide for the preparation of a medicament for increasing the viscosity of mucus layers in mammals; with the proviso that the TFF2 peptide is not glycosylated Lys99-TFF2.

In a third aspect, the present invention relates to a method for in vivo increase in viscosity of mucus layers in a subject, the method comprising administering to the subject a composition comprising

a) a pharmaceutically acceptable carrier or diluent,

b) a therapeutically effective amount of a TFF2 peptide; with the proviso that the TFF2 peptide is not glycosylated Lys99-TFF2,

and optionally

c) a mucin glycoprotein preparation, In another aspect, the present invention relates to the use of a TFF2 peptide for the treatment of conditions with increased viscosity of mucus layers in mammals; with the proviso that the TFF2 peptide is not glycosylated Lys99-TFF2.

The term “treatment”, as used herein, means the administration of an effective amount of a therapeutically active compound of the invention with the purpose of preventing any symptoms or disease state to develop or with the purpose of curing or easing such symptoms or disease states already developed. The term “treatment” is thus meant to include prophylactic and protective treatment. The symptoms or disease state includes but is not limited to diseases, e.g. gastric ulcers or asthma, inherited biological disorders or conditions induced by damaging by external stimuli, e.g. Inhalation of toxic or acidic chemical.

In one embodiment of the invention, the mammal is human.

In another embodiment the present invention relates to a pharmaceutical composition for local application.

In a further embodiment the present invention relates to a pharmaceutical composition for luminal application.

In a further embodiment the present invention relates to a pharmaceutical composition for parenteral administration.

In a further embodiment the present invention relates to a pharmaceutical composition for oral administration.

In a further embodiment the present invention relates to a pharmaceutical composition further comprising a mucin glycoprotein preparation.

In a further embodiment the present invention relates to a pharmaceutical composition for the treatment of oral mucosa.

In a further embodiment the present invention relates to a pharmaceutical composition for the treatment of patients with reduced secretion of saliva. In one embodiment, the reduced secretion of saliva is caused by irradiation therapy, treatment with anticholinergics or Sjögrens syndrome.

In a further embodiment, the present invention relates to a pharmaceutical composition for the treatment of patients receiving irradiation therapy.

In a further embodiment, the present invention relates to a pharmaceutical composition for the treatment of patients treated with anticholinergics.

In a further embodiment, the present invention relates to a pharmaceutical composition for the treatment of patients with Sjögrens syndrome.

In a further embodiment the present invention relates to a pharmaceutical composition for the treatment of the respiratory passages.

In a further embodiment the present invention relates to a pharmaceutical composition for increasing the viscosity of nasal secretions in rhinorrhoea in common cold or allergic rhinitis.

In a further embodiment, the present invention relates to a pharmaceutical composition for the treatment of patients with common cold.

In a further embodiment, the present invention relates to a pharmaceutical composition for the treatment of patients with allergic rhinitis.

In a further embodiment the present invention relates to a pharmaceutical composition for the treatment of the respiratory tract.

In a further embodiment the present invention relates to a pharmaceutical composition for the treatment of the respiratory tract following accidental inhalation of irritants.

In a further embodiment the present invention relates to a pharmaceutical composition for the treatment of the respiratory tract following accidental inhalation of gases, dusts or fumes.

In a further embodiment the present invention relates to a pharmaceutical composition for the treatment of oesophagus. In one embodiment the present invention relates to a pharmaceutical composition for the treatment of the distal part of the oesophagus.

In a further embodiment the present invention relates to a pharmaceutical composition for protection against acid secretions from the stomach.

In a further embodiment the present invention relates to a pharmaceutical composition for protection against acid secretions from the stomach in reflux oesophagus.

In a further embodiment the present invention relates to a pharmaceutical composition for protection against acid secretions from the stomach in hiatus hernia.

In a further embodiment the present invention relates to a pharmaceutical composition for protection against acid secretions from the stomach in Barrets oesophagus.

In a further embodiment the present invention relates to a pharmaceutical composition for the treatment of the stomach.

In a further embodiment the present invention relates to a pharmaceutical composition for treatment of stress induced gastric ulcers. In one embodiment the stress induced gastric ulcers is secondary to trauma. In another embodiment the stress induced gastric ulcers is secondary to shock. In a further embodiment the stress induced gastric ulcers is secondary to large operations. In a further embodiment the stress induced gastric ulcers is secondary to renal diseases. In a further embodiment the stress induced gastric ulcers is secondary to lever diseases. In a further embodiment the stress induced gastric ulcers is secondary to treatment with aspirin, other non-steroidal anti-inflammatory drugs (NSAIDS), steroids or alcohol.

In a further embodiment the present invention relates to a pharmaceutical composition for the treatment of diarrhoea.

In a further embodiment the present invention relates to a pharmaceutical composition for the treatment of the small intestinal mucosa.

In a further embodiment the present invention relates to a pharmaceutical composition for the treatment of the colonic mucosa.

In a further embodiment the present invention relates to a pharmaceutical composition for the treatment of irritable bowel syndrome.

In a further embodiment the present invention relates to a pharmaceutical composition for the treatment of Crohns disease.

In a further embodiment the present invention relates to a pharmaceutical composition for the treatment of ulcerative colitis.

In a further embodiment the present invention relates to a pharmaceutical composition for the treatment of the eye.

In a further embodiment the present invention relates to a pharmaceutical composition for increasing the viscosity of lacrimal fluid.

In a further embodiment the present invention relates to a pharmaceutical composition for increasing the viscosity of lacrimal fluid in patients with keratoconjunctivitis sicca.

In a further embodiment the present invention relates to a pharmaceutical composition for increasing the viscosity of lacrimal fluid in patients with Sjögren's syndrome.

In a further embodiment the present invention relates to a pharmaceutical composition for increasing the viscosity of lacrimal fluid in patients with dry eyes.

The term “dry eyes”, as used herein, means any condition where the eyes feels dry.

In a further embodiment the present invention relates to a pharmaceutical composition in eye droplets.

In a further embodiment the present invention relates to a pharmaceutical composition for the treatment of the knee joints.

In a further embodiment the present invention relates to a pharmaceutical composition for increasing the viscosity of the synovial fluid.

In a further embodiment the present invention relates to a pharmaceutical composition for increasing the viscosity of the synovial fluid in osteoarthritis.

In a further embodiment the present invention relates to a pharmaceutical composition for increasing the viscosity of the synovial fluid following joint replacement.

In a further embodiment the present invention relates to a pharmaceutical composition for the treatment of the bladder.

In a further embodiment the present invention relates to a pharmaceutical composition for the treatment of patients with catheter.

In a further embodiment the present invention relates to a pharmaceutical composition for the treatment of infections. In one embodiment the infection is a cronic infection of the bladder.

In a further embodiment the present invention relates to a pharmaceutical composition for the treatment of interstitial cystitis.

In a further embodiment the present invention relates to a pharmaceutical composition for the treatment of papillomas.

In a further embodiment the present invention relates to a pharmaceutical composition for the treatment of cancer.

In a further embodiment of the invention, the TFF2 peptide is human TFF2.

In a further embodiment of the invention, the TFF2 peptide is recombinant human TFF2.

In a further embodiment of the invention, the TFF2 peptide is human Asn99-TFF2.

In a further embodiment of the invention, the TFF2 peptide is recombinant human Asn99-TFF2.

In a further embodiment of the invention, the TFF2 peptide is glycosylated.

In a further embodiment of the invention, the TFF2 peptide is non-glycosylated.

In a further embodiment of the invention, the TFF2 peptide is glycosylated Asn99-TFF2.

In a further embodiment of the invention, the TFF2 peptide is recombinant glycosylated Asn99-TFF2.

In a further embodiment of the invention, the TFF2 peptide is non-glycosylated Asn99-TFF2.

In a further embodiment of the invention, the TFF2 peptide is recombinant non-glycosylated Asn99-TFF2.

In a further embodiment of the invention, the TFF2 peptide is non-glycosylated Lys99-TFF2.

In a further embodiment of the invention, the TFF2 peptide is recombinant non-glycosylated Lys99-TFF2.

TFF2 peptides are typically produced by recombinant DNA techniques such as described in Danish patent applications no. 2000/01847 and 2000/01850. To this end, a DNA sequence encoding the TFF2 peptide may be isolated by preparing a genomic or cDNA library and screening for DNA sequences coding for all or part of the peptide by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the peptide is preferably of human origin, i.e. derived from a human genomic DNA or cDNA library.

The DNA sequences encoding the TFF2 peptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the TFF2 peptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the TFF2 peptide is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the TFF2 peptide in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Left. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. Nos. 5,155,037; US 5,162,222), or the baculovirus 39K delayed-early gene promoter (U.S. Pat. Nos. 5,155,037; US 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequence encoding the TFF2 peptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may further comprise elements such as polyadenylation signals (e.g. from SV40 or the adenovirus 5 E1b region), transcriptional enhancer sequences (e.g. the SV40 enhancer) and translational enhancer sequences (e.g. the ones encoding adenovirus VA RNAs).

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pvrG, argB, niaD or sC.

To direct a TFF2 peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the TFF2 peptide in the correct reading frame. Secretory signal sequences are commonly positioned 5′ to the DNA sequence encoding the peptide. The secretory signal sequence may be that, normally associated with the peptide or may be from a gene encoding another secreted protein.

For secretion from yeast cells, the secretory signal sequence may encode any signal peptide, which ensures efficient direction of the expressed TFF2 peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the TFF2 peptide. The function of the leader peptide is to allow the expressed peptide to be directed from the endoplasmic reticulum to the Golgi apparatus and further to a secretory vesicle for secretion into the culture medium (i.e. exportation of the TFF2 peptide across the cell wall or at least through the cellular membrane into the periplasmic space of the yeast cell). The leader peptide may be the yeast α-factor leader (the use of which is described in e.g. U.S. Pat. Nos. 4,546,082, US 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the TFF2 peptide, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

The host cell into which the DNA sequence encoding the TFF2 peptide is introduced may be any cell, which is capable of producing the posttranslational modified TFF2 peptide and includes yeast, fungi and higher eucaryotic cells.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigier et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. Nos. 4,599,311, US 4,931,373, US 4,870,008, 5,037,743, and US 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the TFF2 peptide may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. otyzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. Nos. 4,745,051; US 4,879,236; US 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the TFF2 peptides after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The TFF2 peptides produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

In the pharmaceutical composition of the invention, the TFF2 peptides may be formulated by any of the established methods of formulating pharmaceutical compositions, e.g. as described in Reminqton's Pharmaceutical Sciences, 1985. The composition may be in a form suited for systemic injection or infusion and may, as such, be formulated with sterile water or an isotonic saline or glucose solution. The compositions may be sterilized by conventional sterilization techniques, which are well known in the art. The resulting aqueous solutions may be packaged for use or filtered under aseptic conditions and lyophilized, the lyophilized preparation being combined with the sterile aqueous solution prior to administration. The composition may contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

The pharmaceutical composition of the present invention may also be adapted for nasal, transdermal or rectal administration. The pharmaceutically acceptable carrier or diluent employed in the composition may be any conventional solid carrier. Examples of solid carriers are lactose, terra alba, sucrose, talc, gelatin, agar, pectin, acacia, magnesium stearate and stearic acid. Similarly, the carrier or diluent may include any sustained release material known in the art, such as glyceryl monostearate or glyceryl distearate, alone or mixed with a wax. The amount of solid carrier will vary widely but will usually be from about 25 mg to about 1 g.

The concentration of the TFF2 peptides in the composition may vary widely, i.e. from from about 5% to about 100% by weight. A typical concentration is in the range of 50-100% by weight. A unit dosage of the composition may contain from about 1 mg to about 200 mg, typically from about 25 mg to about 75 mg, such as about 50 mg, of the peptide.

The term “a therapheutically effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment (e.g. trauma, ulcerative colitis, gastric ulcers), patient-related factors (e.g. weight, health, age, etc.), presence of co-administered medications, time of administration, or other factors known to a medical practitioner. The dosage of a TFF2 peptide administered to a patient will vary with the type and severity of the condition to be treated, but is generally in the range of 0.1-1.0 mg/kg body weight.

The term “subject” as used herein is intended to mean any animal, in particular mammals, such as humans, and may, where appropriate, be used interchangeably with the term “patient”.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Example 1

Construction of a Yeast Expression System for LYS99-TFF2.

A Saccharomyces cerevisae expression system expressing a mutant hSP with an Asn at position 99 of the mature protein (hSP-Asn₉₉) has been described previously (Thim, L., 1993, FEBS Letters 318: 345-352).

The yeast plasmid called pKFN-1847 (Thim et al., FEBS Letters, 1993, 318: 345-352) contains an expression cassette comprising an EcoRI-XbaI DNA fragment inserted into the plasmid between the transcription-promoter (located on a SalI-EcoRI fragment) and the transcription-terminator of the Saccharomyces cerevisiae TPI1 gene.

In plasmid pKFN-1847 the EcoRI-XbaI fragment encodes a fusion protein composed of a leader sequence, a Lys-Arg cleavage site for the dibasic processing endopeptidase KEX2, and hSP-Asn₉₉. In order to construct a plasmid encoding Lys99-TFF2, the following steps were performed using standard molecular biology techniques (e.g. Sambrook, J., Fritsch, E. F. and Maniatis, T., Molecular Cloning: A laboratory Manual, Cold Spring Harbour Laboratory Press, New York, 1989).

A 688 bp DNA fragment containing the EcoRI-XbaI DNA fragment and encoding the leader-hSP fusion protein was amplified with PCR from plasmid pKFN-1847 using oligonucleotides EA-ECO: (5′-CTA TTT TCC CTT CTT ACG-3′) and El 47: (5′-TM TCT TAG TTT CTA GAC TTA GTA ATG GCA GTC TCT CAC AGA CTT CGG GM GM GC -3′). EA-ECO corresponds to a sequence located 114 bp upstream from the EcoRI site of the EcoRI-XbaI DNA fragment containing the expression cassette. E147 has been designed to introduce a single nucleotide mutation in the DNA sequence encoding hSP-Asn₉₉ changing Asn₉₉ of hSP-Asn₉₉ to Lys₉₉. After digestion with EcoRI and XbaI the DNA sequence encoding hSP-Lys₉₉, hereafter referred to as Lys99-TFF2, can thus be cloned as a EcoRI-XbaI DNA fragment.

The EcoRI-XbaI PCR fragment containing the DNA sequence encoding the leader-Lys99-TFF2 fusion protein was ligated to the ApaI-EcoRI DNA fragment of pMT742 (Egel-Mitani et al., Gene, 1988, 73: 113-120) containing the TPI1 promoter from S. cerevisiae and the ApaI-XbaI vector fragment of pMT742, resulting in plasmid pEA314. The plasmid pMT742 has a similar organization as pKFN-1847.

The expression plasmid was propagated in E. coli, grown in the presence of ampicillin and isolated using standard techniques (Sambrook et al., 1989). The plasmid DNA was checked for insert by appropriate restriction nucleases (e.g. EcoRI, NcoI, ApaI, XbaI) and was shown by sequence analysis to contain the proper DNA sequence encoding Lys99-TFF2.

The plasmid pEA314 was transformed into S. cerevisiae strain MT663. Yeast transformants harbouring plasmid pEA314 were selected by glucose utilization as carbon source on YPD (1% yeast extract, 2% peptone, 2% glucose) agar (2%) plates. One transformant yEA314, was selected for fermentation.

Yeast strain yEA314 was cultivated at 30° C. for 72 hours in YPD media (Guthrie, C. & Fink, G. R., Eds., Guide to Yeast Genetics and Molecular Biology, Academic Press, 1991) with a final OD₆₀₀ of approximately 15-20. After centrifugation the cell pellet was discarded and the supernatant was used for further characterization of Lys99-TFF2.

S. cerevisiae strain MT663 (MATa/MATα pep4-3/pep4-3 HIS4/his4 tpi::LEU21tpi::LEU2 Cir⁺) was used as host strain for transformation. Strain MT663 was deposited in the Deutsche Sammiung von Mikroorganismen und Zellkulturen in connection with filing WO 92/11378 and was given the deposit number DSM 6278. Transformation of MT633 was conducted as described in WO 98/01535

### Example 2

Purification of LYS99-TFF2

Yeast fermentation supernatant from yEA314 was concentrated from 2.5 ml to 0.25 ml using a Centricon® YM-3 3000 centrifugal filter device with the method described by the manufacturer (Millipore Corporation). The concentrated sample (0.25 ml) was injected onto a Vydac 214TP54 reverse-phase C4 HPLC column (0.46×25 cm) equilibrated at 25° C. at a flow rate of 1.0 ml/min with 0.1% (v/v) Trifluoroacetic acid in 10% (v/v) acetonitrile. After isocratic elution in 10 min the concentration of acetonitrile in the eluting solvent was raised to 60% (v/v) over 25 minutes. Absorbance was measured at 214 nm. The peaks eluting at 27.254 min. and 28.038 min. was found by mass spectrometry analysis to represent glycosylated human Lys99-TFF2 and non-g lycosylated human Lys99-TFF2, respectively.

### Example 3

Rheological Properties of TFF2 Peptides

Three types of mucins were investigated. Mucin I: Crude mucin, type 11 from porcine stomach (Sigma, St. Louis, Mo., USA). Mucin II: Partially purified mucin, type III from porcine stomach (Sigma, St. Louis, Mo., USA). Mucin III: mucin, type I-S from bovine submaxillary glands (Sigma, St. Louis, Mo., USA). TFF2 only exist in a dimer form (FIG. 1). Recombinant human Asn99-TFF2 in both glycosylated and the non-glycosylated forms were prepared in a yeast expression system as previously described (Thim, L. et al. (1993) FEBS Lett. 318, 345-352).

Mucin solutions. A 10% (w/v) solution of mucin I was prepared and different trefoil peptides were dissolved in water and added to the mucin solution. After mixing the sample (Vortex mixer), the sample was allowed to stand for 5 min. and the viscosity was visually assessed in relation to a control solution of mucin added water without TFF2. The detailed experimental conditions for the rheometer measurement are given in the figure legends.

Rheological measurements. Rheological properties were measured by the use of a rotational Reologica Rheometer (Reologica Instriments AB, Lund, Sweden). The instrument is equipped with a stainless steel C40 4 cone-plate (40 mm diameter plate with an angle of 4 degree) requiring a sample volume of at least 1.2 ml. The instrument was operated using instrument standard software (Version 3.6) allowing several different types of measurements. In the present study we have used the measuring programs: Constant Rate (viscosity and stress as a function of shear rate), Oscillation (complex viscosity, elastic modulus and viscous modulus at different frequencies) and Oscillation Stress Sweep (to identify the stress range inside which the measurement results are linear i.e. independent of the applied stress).

A visual assessment of the change in properties that could be observed when different TFF2 peptides was added to mucin solutions was made (Table 1). In some experiments the effect was astonishing. The addition of TFF2 peptides to mucin solutions resulted in thick gel-like substance that did hardly leave the test-tube even if the tube was turned bottom up.

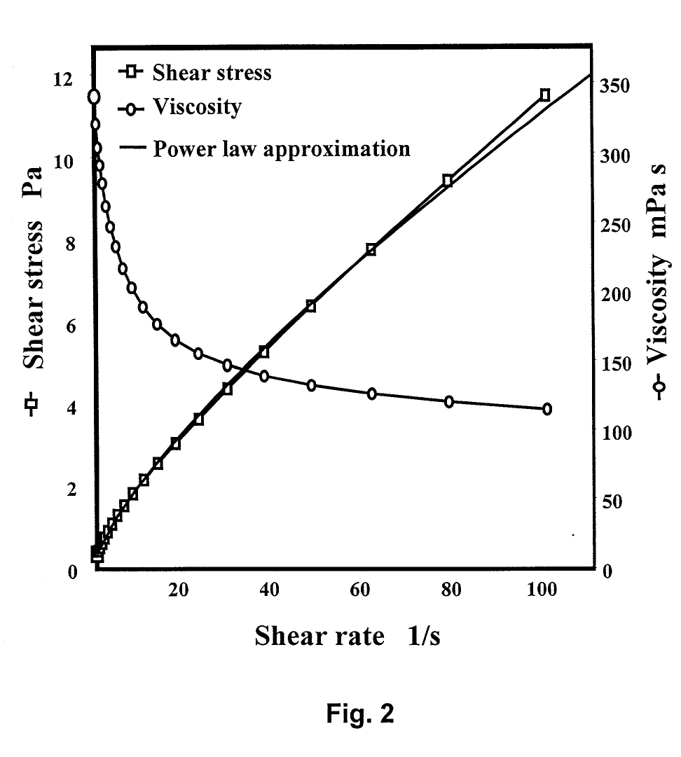

Mucin solutions and mucin/TFF2 gel-like substances. Mucin solutions to which a TFF2 peptide was added was compared. As can be seen from FIG. 2 the mucin solution alone behaves as a non-Newtonian liquid. These liquids can be described by the Ostwald de Waele model (power law) (Barnes, H. A. (1989) An introduction to rheology. Elsevier and Ferguson, J. and Kemblowski, Z. (1991) Applied fluid rheology. Elsevier):

δ=k(γ)ⁿ,

where δ=shear stress, γ=shear rate and n and k are constants specific for the solution (if n=1 the solution is Newtonian). In the present case the following values could be calculated from FIG. 2: n=0.75 and k=0.35.

Since n<1 the solution is called shear-tinning, which is the characteristics of dispensions with asymmetric particles or emulsions. However, since the n value is close to 1 the solution is not far from being Newtonian. As can also be seen from FIG. 2 the viscosity varies from 0.34 Pa s at low shear rates to 0.12 Pa s at high shear rates.

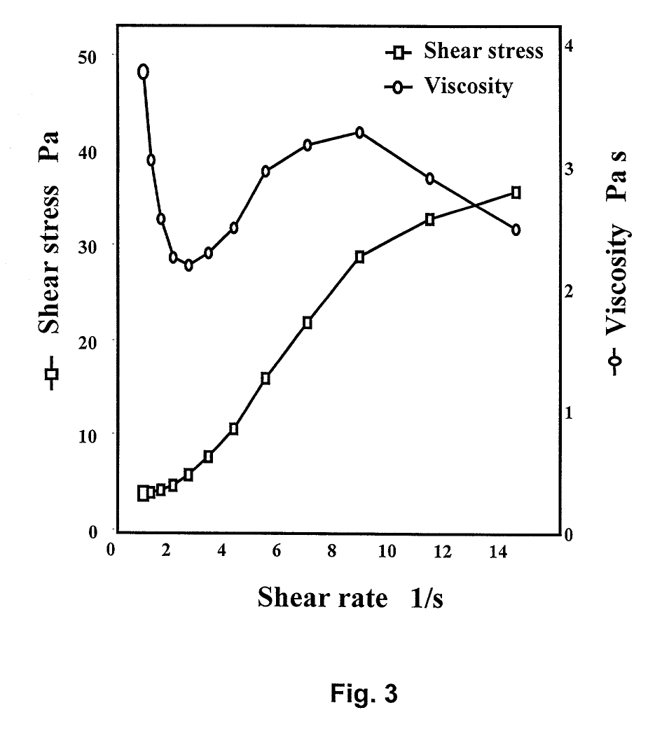

FIG. 3 shows the stress versus shear rate measurement of the mucin/TFF2 solution. As compared to the mucin solution alone (FIG. 2) a rather dramatically change occurred by the addition of the TFF2 peptide. The viscosity increased from 0.12-0.35 Pa s (FIG. 2) to 2.2-3.7 Pa s (FIG. 3) i.e. more than a factor 10 and the shear stress increased e.g. from 1.8 to 29 Pa at a shear rate of 9 s⁻¹.

As could also be visually observed the mucin/TFF2 forms a gel-like structure and thus behaves like a viscoelastic material.

In order to characterise the mucin/TFF2 gel-like structure, the technique of oscillatory measurement in which the gel-like material are subjected to a sinusoidally varying stress was applied and the strain response was measured. Before this measurement was carried out we used an oscillation stress sweep programme to define the so-called linear viscoelastic region. Inside this region no change of the mucin/TFF2 structure occurs and the relation between the applied stress and the measured quantities is linear. The oscillation sweep test showed that an applied stress corresponding to 0.1 Pa was suitable for the mucin/TFF2 gel-like material and the subsequent oscillatory measurement was therefore carried out using this value and a frequency interval from 0.01 to 5 Hz.

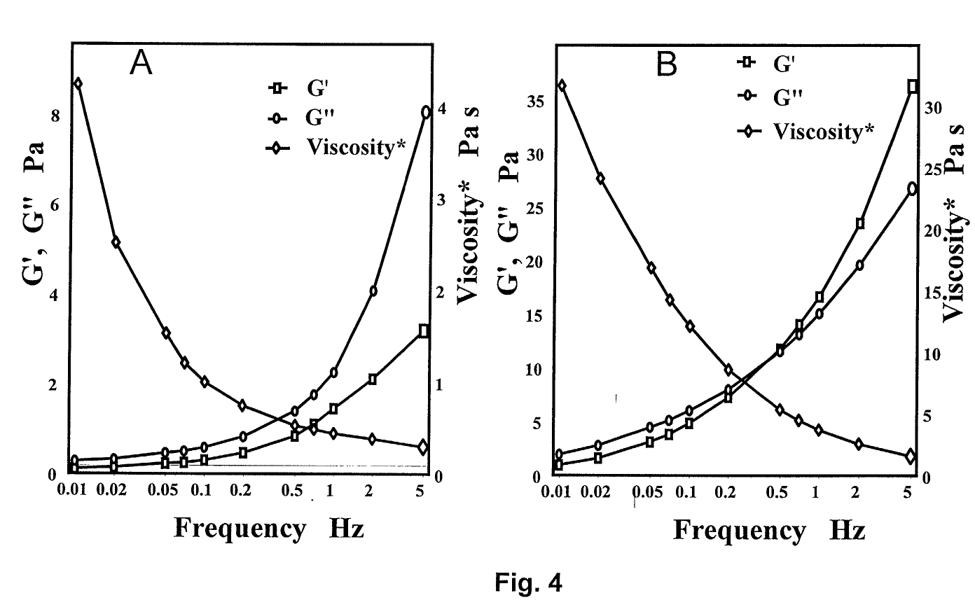

FIG. 4 shows the result from the oscillatory measurement of the mucin solution alone (FIG. 4a) and the mucin/TFF2 gel-like material (FIG. 4b). This type of experiments allow the estimation of several rheological parameters as a function of frequency: complex viscosity η*, elastic modulus G′ and viscous modulus G″ G″ (for detailed rheological theory see Barnes, H. A. (1989) An introduction to rheology. Elsevier and Ferguson, J. and Kemblowski, Z. (1991) Applied fluid rheology. Elsevier)

A comparison of the absolute values of the elastic and viscous moduli of the mucin solution and the mucin/TFF2 gel-like material is given in table 2. As can be seen from these results both the elastic modulus and the viscous modulus are dramatically increased in the mucin/TFF2 gel-like structure as compared to the mucin solution.

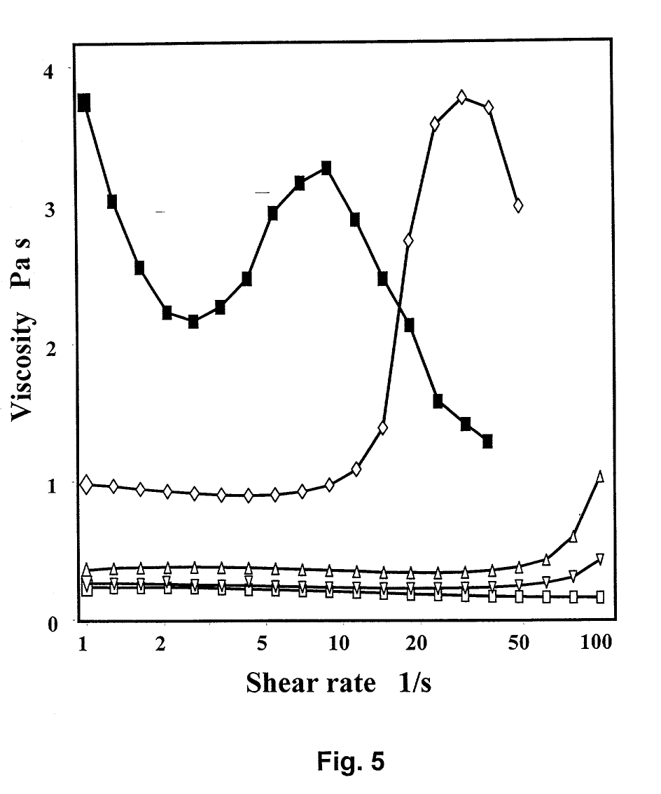

TFF2 peptide concentration. In order to form the highly viscous mucin/TFF2 gel-like structure a certain amount of TFF2 peptide is needed. FIG. 5 shows an experiment in which increasing amount of TFF2 peptide was added to 2 ml of a 10% (w/v) mucin I solution. By the addition of the 0.88 mg, 1.76 mg and 3.53 mg glycosylated TFF2, respectively no major change in the viscosity was seen. However the addition of 7.05 mg (or 14.1 mg) of the TFF2 peptide resulted in the mucin/TFF2 gel-like structure to be formed (FIG. 5). So in this experimental set up the critical TFF2 peptide amount is between 3.53 and 7.05 mg per 2 ml 10% mucin solution. This corresponds to a ration of TFF2 peptide to mucin of 1.8-3.6% (w/w).

Mucin type and concentration. A fixed amount of TFF2 peptide (7.05 mg glycosylated TFF2) was added to 2 ml X % (w/v) mucin I solution in the experimental set up described in the legend to FIG. 5. The X % was varied from 6%, 8%, 10%, 12% and 14% (w/v). No mucin/TFF2 gel-like structure was formed with the 6% and 8% mucin solution, but a fibre-like precipitate surrounded by liquid mucin solution was formed. Using the 10%, 12% and 14% mucin solution the mucin/TFF2 gel-like structure (FIGS. 3 and 4) was formed.

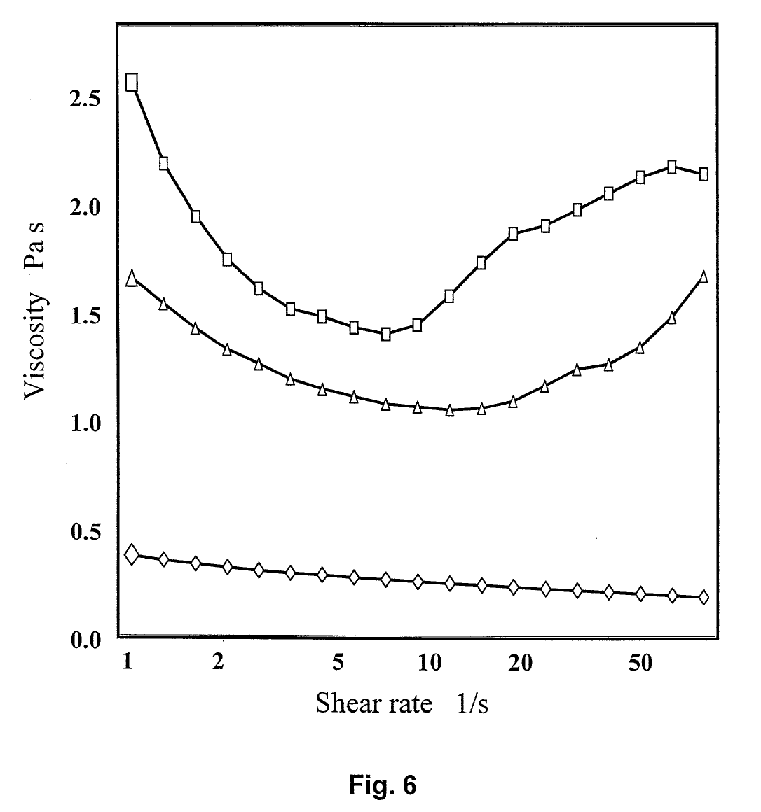

FIG. 6 shows the result obtained with 3 different mucin types. Both mucin type I and II formed the mucin/TFF2 gel-like structure. Mucin I and II are both from porcine stomach. Mucin III is from bovine submaxillary gland.

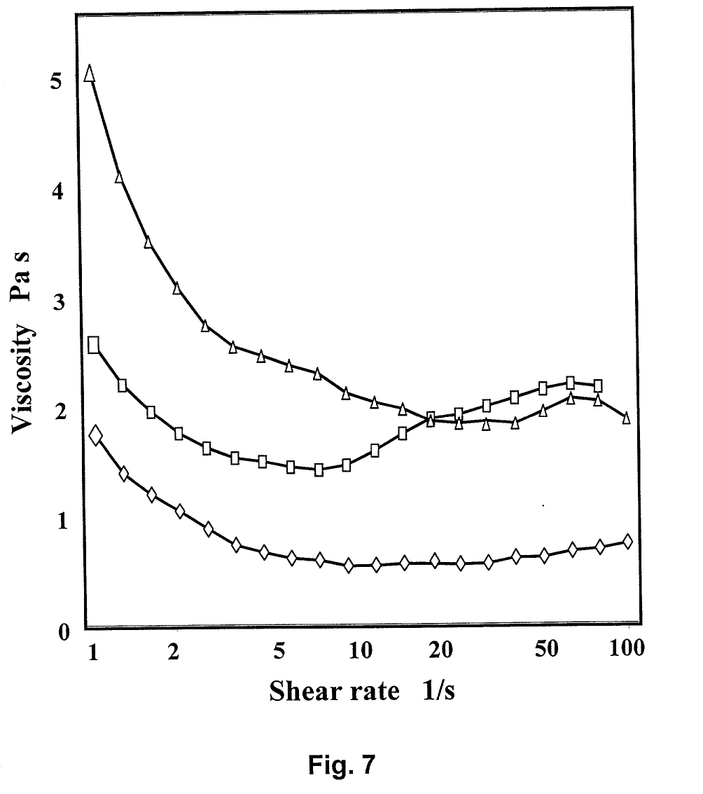

pH dependence. FIG. 7 shows the formation of the mucin/TFF2 gel-like structure at three different conditions. The most viscous gel is formed in 0.01 N HCl. At high shear rates the structure formed in water and in 0.01 N HCl has nearly identical viscosity both being more viscous that the gel-like material formed at neutral pH.

Glycosylation of the TFF2 peptide. Both Lys99-TFF2 and Asn99-TFF2 were prepared in a glycosylated and non-glycosylated form. These four TFF2 peptides were compared in a system similar to the one described in the legend to FIG. 5 (2 ml of 10% (w/v) mucin I dissolved in 0.05% (w/v) sodiumazide was added 0.4 ml of water containing 7.05 mg of the TFF2 peptide in question). All four TFF2 peptides were able to form highly viscous mucin/TFF2 gel-like structures. However the two glycosylated forms seem to generate a more viscous mucin/TFF2gel-like structure that the non-glycosylated forms but there was no difference between the glycosylated Lys99-TFF2 and the glycosylated Asn99-TFF2. For example in a system where the glycosylated Asn99-TFF2 had a viscosity of 0.8 Pa s the corresponding non-glycosylated Asn99-TFF2 only had a viscosity of 0.54 Pa s.

TABLE 1
Visual assessment of viscosity

| | | Amount TFF2 | Viscosity |
|---|---|---|---|
| Mucin I solution | TFF2 peptide | added | increase |
| 1 ml 10%(w/w) | Lys99-TFF2 | 10 mg in 200 μl | +++++ |
| 1 ml 10%(w/w) | Asn99-TFF2 | 7.5 mg in 200 μl | +++++ |
| 1 ml 10%(w/w) | Glycosylated Asn99- | 7.1 mg in 200 μl | +++++ |
| | TFF2 | | |

TABLE 2
Elastic and viscous modules of mucin solution as compared
to the mucin solution after TFF2 addition (Experimental
details is given in legend to FIG. 4).

| | Frequency | | | | | |
|---|---|---|---|---|---|---|
| | 0.01 Hz | | 0.1 Hz | | 1 Hz | |
| | | Mucin/ | | Mucin/ | | Mucine/ |
| Material | Mucin | TFF2 | Mucin | TFF2 | Mucin | TFF2 |
| Elastic | 0.08 | 0.8 | 0.27 | 4.7 | 1.4 | 17 |
| modules | | | | | | |
| G′ (Pa) | | | | | | |
| Viscous | 0.25 | 1.8 | 0.55 | 5.9 | 2.2 | 15 |
| modulus | | | | | | |
| G″ (Pa) | | | | | | |

### Example 4

The effect of Luminal Asn99-TFF2 in Experimental Colitis in Rats

Methods:

16 female wistar rats weighing 200 g were used in the study. The effect of luminal Asn99-TFF2 was investigated in a rat model of colitis - the dextran induced colitis model (Mottet N K. Gastroenterology 1972;62:1269-71). Asn99-TFF2 was administrated directly into the proximal part of the colon by means of a soft polyethylene tube, which in anaesthezised rats at a laparotomy was inserted into the colonic lumen, secured by 6-0 silk sutures and lead subcutaneously to the neck region of the rat. After this operation the rats had a recovery period of 6 days.

In the 16 rats colitis was induced by means of dextran sulphate sodium 5% administered in the drinking water. Eight of the rats were given Asn99-TFF2, 5 mg/kg in 0.5 ml H₂O into the colonic tube, two times each day from the day before the initiation of dextran supplementation till day 9, where the rats were sacrificed. The 8 controls recieved NaCl.

The rats were sacrificed by means of an overdose of barbiturate. The colon was fixed by intraluminal injection of 10% formalin, and after 10 minutes opened and suspended on a polyethylene plate. After further 24 hours fixation the specimens were flushed with water and surface stained with 0.3% Alcian Green 3BX. The colonic specimens were investigated by means of a Wild Photomacroscope—the extent of disease and the number of ulcerations were quantitated.

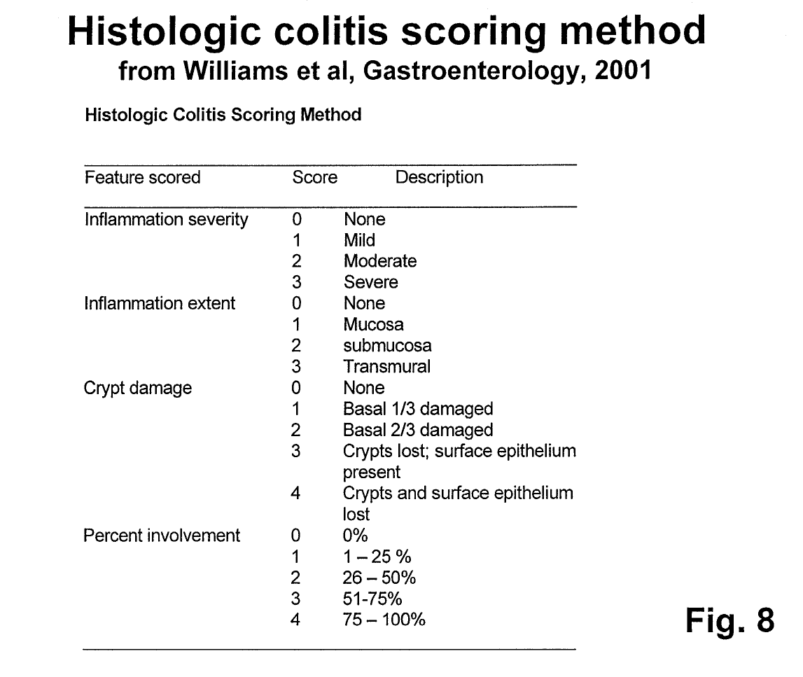

For histologic analysis specimens were taken out (in a blinded way) from the proximal, middle and distal colon and embedded in paraffin. Histologic sections of 5 μm were stained with PAS-hematoxylin-aurentia. The severety of colitis was scored by means of a histologic colitis score (FIG. 8, Williams K L. et al. Gastroenterology 2001;120:925-37).

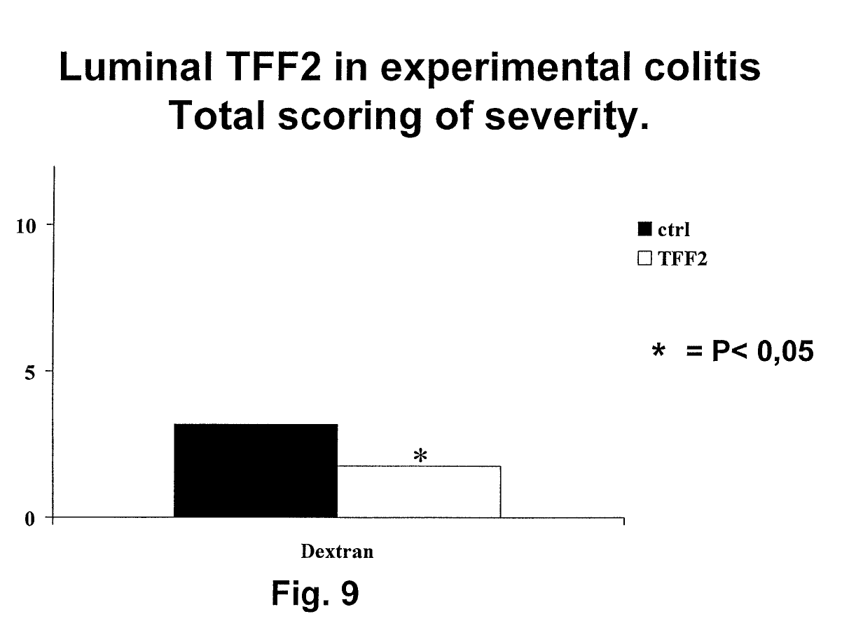

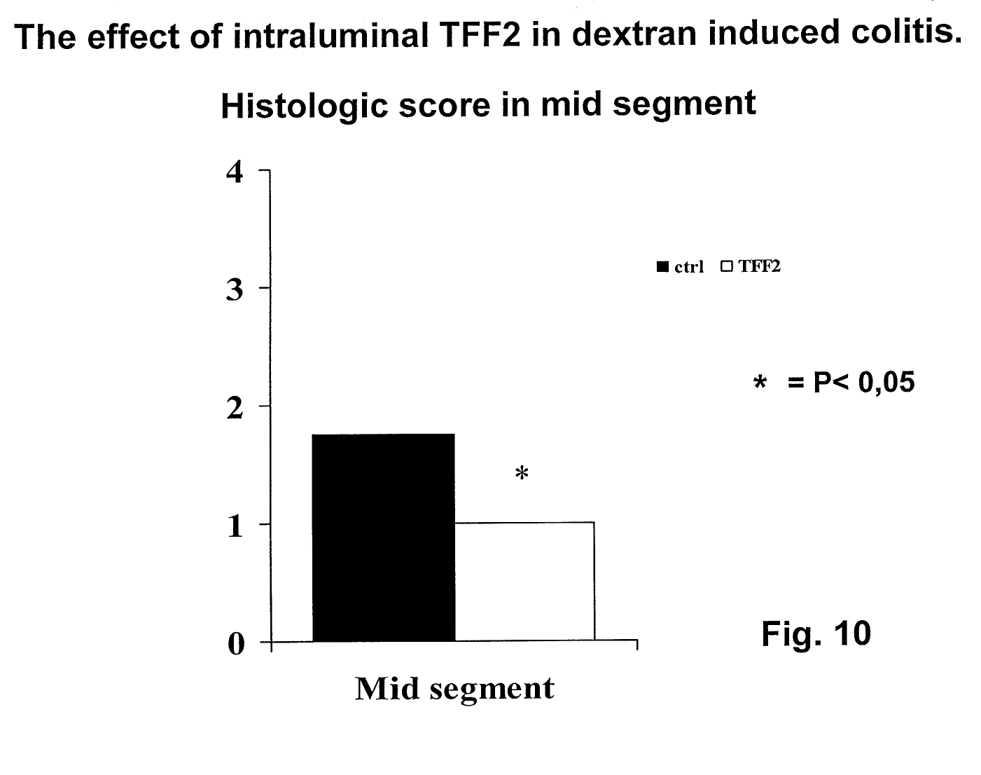

Results: Intraluminal treatment with Asn99-TFF2 had a significant effect on the overall colitis score (1.8 v.s.2.3; p<0.05, FIG. 9). The effect was predominantly in the mid-section of the colon close to the site where the Asn99-TFF2 had been introduced into the colonic lumen (1.0 vs 1.8; p<0.05, FIG. 10).

Conclusion: Intraluminal treatment with Asn99-TFF2 reduces the severity of dextran induced colitis in the rat.

1. A pharmaceutical composition for increasing the viscosity of mucus layers in mammals, the composition comprising a TFF2 peptide or a pharmaceutically acceptable salt thereof; with the proviso that the TFF2 peptide is not glycosylated Lys99-TFF2.

2. The pharmaceutical composition according to claim 1, wherein the mammal is human.

3. The pharmaceutical composition according to any one of claims 1-2 for local and luminal application.

4. The pharmaceutical composition according to any one of claims 1-2 for parenteral administration.

5. The pharmaceutical composition according to any one of claims 1-2 for oral administration.

6. The pharmaceutical composition according to any one of claims 1-5, wherein the TFF2 peptide is recombinant human TFF2.

7. The pharmaceutical composition according to claim 6, wherein the human TFF2 peptide is glycosylated.

8. The pharmaceutical composition according to any one of claims 1-7, wherein the composition further comprises a mucin glycoprotein preparation.

9. The pharmaceutical composition according to any one of claims 1-8, for the treatment of oral mucosa.

10. The pharmaceutical composition according to claim 9, for the treatment of patients with reduced secretion of saliva.

11. The pharmaceutical composition according to claim 10, wherein the reduced secretion of saliva is caused by irradiation therapy, treatment with anticholinergics or Sjögrens syndrome.

12. The pharmaceutical composition according to any one of claims 1-8, for the treatment of the respiratory passages.

13. The pharmaceutical composition according to claim 12, for increasing the viscosity of nasal secretions in rhinorrhoea in common cold or allergic rhinitis.

14. The pharmaceutical composition according to claim 13, for the treatment of the respiratory tract following accidental inhalation of irritants, gases, dusts or fumes.

15. The pharmaceutical composition according to any one of claims 1-8, for the treatment of the distal part of the oesophagus.

16. The pharmaceutical composition according to claim 15, for protection against acid secretions from the stomach in reflux oesophagus, hiatus hernia or Barrets oesophagus.

17. The pharmaceutical composition according to any one of claims 1-8, for the treatment of the stomach.

18. The pharmaceutical composition according to claim 17, for treatment of stress induced gastric ulcers secondary to trauma, shock, large operations, renal or lever diseases, or treatment with aspirin, other NSAIDS, steroids or alcohol.

19. The pharmaceutical composition according to any one of claims 1-8, for the treatment of diarrhoea.

20. The pharmaceutical composition according to any one of claims 1-8, for the treatment of the small intestinal or colonic mucosa in Crohns disease, ulcerative colitis or irritable bowel syndrome.

21. The pharmaceutical composition according to any one of claims 1-8, for the treatment of the eye.

22. The pharmaceutical composition according to claim 21, for increasing the viscosity of lacrimal fluid in patients with keratoconjunctivitis sicca/Sjögren's syndrome or dry eyes.

23. The pharmaceutical composition according to any one of claims 21-22, wherein the pharmaceutical composition is in eye droplets.

24. The pharmaceutical composition according to any one of claims 1-8, for the treatment of the knee joints.

25. The pharmaceutical composition according to claim 24, for increasing the viscosity of the synovial fluid in osteoarthritis and following joint replacement.

26. The pharmaceutical composition according to any one of claims 1-8, for the treatment of chronic bladder infections, patients with catheter, interstitial cystitis, papillomas or cancer of the bladder.

27. Use of a TFF2 peptide for the preparation of a medicament for increasing the viscosity of mucus layers in mammals; with the proviso that the TFF2 peptide is not glycosylated Lys99-TFF2.

28. Use of a TFF2 peptide for the preparation of a medicament for increasing the viscosity of mucus layers in mammals, wherein the medicament is according to any one of the claims 1-26.

29. Use according to any one of the claims 27-28, wherein the mammal is human.

30. A method for increasing viscosity of mucus layers in a subject, said method comprising administering to the subject a composition comprising
a) a pharmaceutically acceptable carrier or diluent,
b) a therapeutically effective amount of a trefoil factor 2 (TFF2) peptide or a salt thereof; with the proviso that the TFF2 peptide is not glycosylated Lys99-TFF2,
and optionally
c) a mucin glycoprotein preparation.

31. The method according to claim 30, wherein the administration is local and luminal.

32. The method according to claim 30, wherein the administration is parenteral.

33. The method according to claim 30, wherein the TFF2 peptide is recombinant human TFF2.

34. The method according to claim 33, wherein the TFF2 peptide is glycosylated.

35. The method according to claim 30, wherein the viscosity of the mucus layers is associated with a disease state in the oral mucosa.

36. The method according to claim 35, wherein the disease state is a reduced secretion of saliva.

37. The method according to claim 36, wherein the reduced secretion of saliva is caused by irradiation therapy, treatment with anticholinergics or Sjögrens syndrome.

38. The method according to claim 30, wherein the viscosity of the mucus layers is associated with a disease state in the respiratory passages.

39. The method according to claim 38, wherein the disease state is nasal secretions in rhinorrhoea in common cold or allergic rhinitis.

40. The method according to claim 38, wherein the disease state is accidental inhalation of irritants, gases, dusts or fumes.

41. The method according to claim 30, wherein the viscosity of the mucus layers is associated with a disease state in the distal part of the oesophagus.

42. The method according to claim 41, wherein the disease state is acid secretions from the stomach in reflux oesophagi's, hiatus hernia or Barrets oesophagus.

43. The method according to claim 30, wherein the viscosity of the mucus layers is associated with a disease state in the stomach.

44. The method according to claim 43, wherein the disease state is stress induced gastric ulcers secondary to trauma, shock, large operations, renal or lever diseases, or treatment with aspirin, other non-steroidal anti-inflammatory drugs (NSAIDS), steroids or alcohol.

45. The method according to claim 30, wherein the disease state is diarrhoea.

46. The method according to claim 30, wherein the viscosity of the mucus layers is associated with a disease state in the small intestine or colon.

47. The method according to claim 46, wherein the disease state is Crohns disease, ulcerative colitis or irritable bowel syndrome.

48. The method according to claim 30, wherein the viscosity of the mucus layers is associated with a disease state in the eye.

49. The method according to claim 48, wherein the disease state is keratoconjunctivitis sicca/Sjögren's syndrome or dry eyes.

50. The method according to claim 30, wherein the viscosity of the mucus layers is associated with a disease state in the knee joints.

51. The method according to claim 50, wherein the disease state is increased viscosity of the synovial fluid in osteoarthritis or following joint replacement.

52. The method according to claim 30, wherein the disease state is chronic bladder infections, patients with catheter, interstitial cystitis, papillomas or cancer of the bladder.
